(19) United States
(12) Patent Application Publication
(10) Pub. No.: US 2023/0301812 A1
(43) Pub. Date: Sep. 28, 2023

(54) STIRRUP FOR USE IN AN ANKLE FOOT ORTHOSIS

(71) Applicant: Launchpad O &P LLC, Minneapolis, MN (US)

(72) Inventor: Patrick Scott Hinshon, Maplewood, MN (US)

(21) Appl. No.: 18/189,063

(22) Filed: Mar. 23, 2023

Related U.S. Application Data
(60) Provisional application No. 63/269,798, filed on Mar. 23, 2022.

(51) Int. Cl. A61F 5/01

(52) U.S. Cl. CPC A61F 5/0111

(57) ABSTRACT

A stirrup footplate made up of a plastic main body having a predetermined thickness and a generally elongated configuration extending along a main axis. The main body has a toe end with a generally extending below a forefoot of the user when bonded to an AFO, and a heel end with a cross-like configuration, wherein the cross-like configuration has a pair of lateral extensions extending substantially perpendicularly from the main axis. The lateral extensions sized so that an end portion of each lateral extension will be formable in a manner to extend up an opposite side of the thermoplastic AFO when bonded thereto. The cross-like configuration further having a heel extension which extends along the main axis and can extend at least partially up a heel side of the AFO when bonded thereto. The plastic main body is form of a material having a predetermined stiffness which is stiffer than the thermoplastic AFO which will provide support to the user's heel and foot bed.

### BACKGROUND

Many different devices have been utilized in the past to assist with lower extremity conditions that lead to weakness, abnormal gait, contracture, and joint deformity. One of these devices is an ankle foot orthosis (AFO), which helps provide support and assist in normalizing gait mechanics, balance and stability, and endurance and distance. AFOs normalize gait by providing support for joint segments of the foot, ankle and knee with stop motion or resistance. AFOs come in many different forms, material selections, trimlines, strap configurations and components to provide very specific functions to the patient.

As is well known, traditional AFOs typically combine multiple materials to achieve the necessary support functions, while also providing comfort and usability. That said, the selection configuration and characteristics of the various components involved greatly affect the way an AFO operates, and consequently need to be carefully considered when designing such products.

One particular concern with AFO design is the flexibility of the footplate. When the footplate is too flexible, the patient has decreased power at toe-off and it does not generate propulsion late in the stance cycle to achieve adequate step length on the contralateral swing limb. Footplate stiffness will provide various level of stability for the user, and thus should be carefully designed into the AFO.

To provide specific levels of stability and other functions, early braces (early AFOs) used a metal stirrup which is incorporated into a shoe. These braces, as generally illustrated in FIG. 1, typically included metal components and leather shoes, much like the ones made famous by Forrest Gump, which are now ingrained in the visual memory of many. In these devices, a stirrup component 12 is placed on or within the sole 14 of a shoe 16 and has side “uprights” 18 on either side of the user's ankle. At an upper end point 20 of stirrup component 12 is an attachment structure 22 connecting to a longer medial and lateral upright 24. This attachment structure 22 serves as a double action hinge controlling motion in the sagittal plane. At the top of the uprights 24 is a calf band 26 that is secured to the user's leg with a closure 28 (buckle or velcro strap).

As can be appreciated, it would be beneficial to have an orthosis that is more streamlined and user friendly that can be used in many different shoes rather than being attached to a specific shoe. Further, thermoplastic AFO designs provide additional support while increasing surface area on the limb, thus providing enhanced foot and ankle alignment control, and more comfort for the user. Further, the traditional AFO, as generally discussed above, does not provide support beyond creating a structured ankle joint. More specifically, the prior art AFO is not capable of providing any significant lateral support to the user. As such, an improved and more efficient AFO is clearly desirable.

### SUMMARY

An improved ankle foot orthosis (AFO) is achieved by incorporating a reinforced stirrup footplate into the design. The reinforced stirrup footplate is specifically configured to have desired levels of stiffness and is structured in a way simulate pushoff within a normal gait cycle. The additional stiffness below a user's foot, and support on either side of the user's heel also controls compensations in the transverse plane (in-toeing and out-toeing). This stirrup footplate is also configured to be easily incorporated into an AFO in a manner that provides significant levels of support, in multiple directions, thereby maximizing the functional benefit to the patient.

### BRIEF DESCRIPTION OF THE DRAWINGS

Further advantages and features of the various embodiments can be seen from reading the following description, in conjunction with the drawings in which:

FIG. 1 is a sideview of a prior art AFO, well recognized by those skilled in the art;

FIG. 2 is a top view of a stirrup footplate;

FIG. 3 is the sideview of an AFO, incorporating the stirrup footplate into its design;

FIG. 4 is a bottom perspective view of the AFO, further illustrating the stirrup footplate as incorporated into an AFO;

FIG. 5 is a bottom view of the AFO, showing the placement and configuration of the stirrup footplate;

FIG. 6 is a sideview of an AFO;

FIG. 7 is a front view of the AFO;

FIG. 8 is a second sideview of the AFO;

FIG. 9 is a rearview of the AFO;

FIG. 10 is a sideview of the AFO as inserted into a shoe; and

FIG. 11 is a sideview of the AFO inserted into a shoe, with the shoe being laced/closed.

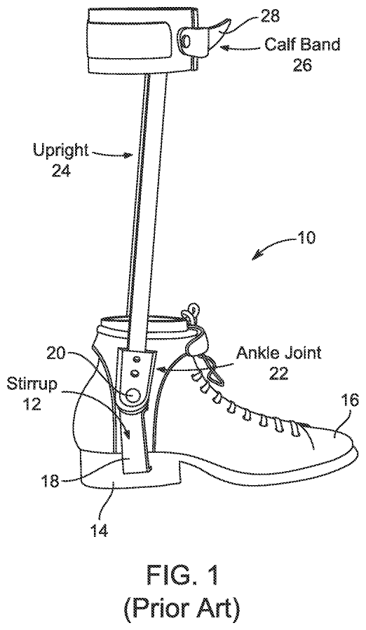

### DESCRIPTION

To provide additional levels of stability for users of an AFO, a simplified stirrup footplate is being provided which can be formed into the plantar surface of an AFO to provide additional levels of stability in numerous directions. A primary goal of this simplified stirrup footplate is to provide stability along the length of the footplate, while also providing lateral or torsional stability for the user's foot while in use. As will be described in further detail below, the stirrup foot plate is an accessory component that is fabricated and bonded to the plantar surface of a custom fabricated thermal plastic AFO.

A rigid/reinforced foot plate is often indicated for patients that have sagittal plane deficits that include crouch gait, early heel off, fall-off at the knee at mid-stance, anterior center of mass, toe walking, plantarflexion weakness, and observed short step length. AFO stiffness provides additional control, mid-stance and terminal stance and encourages knee extension by resisting ankle dorsiflexion. The increased stiffness in the toe plate creates a ground reaction force that encourages knee extension, propulsion at push-off and contralateral increase step length.

The various embodiments of the stirrup footplate discussed in further detail below includes lateral projections that are located at approximately malleolar center while viewing in the sagittal plane. They extend proximally just distal to the malleolus mediately and laterally. These lateral extensions increase transverse plane and coronal plane stiffness, preventing commonly observed, external and internal foot projections (torsional rotation) and genuvalgum in the coronal plane.

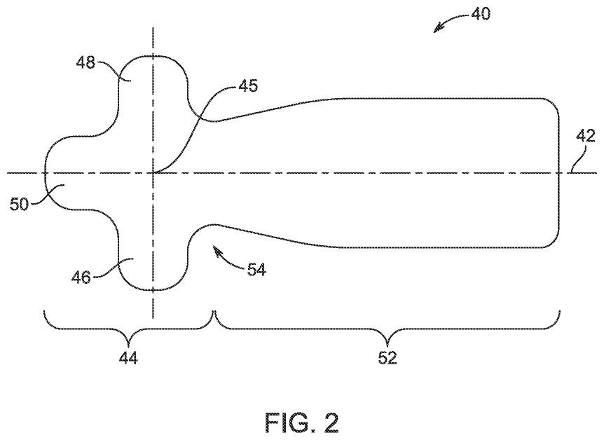

Turning now to FIG. 2, a top view of one example embodiment of a simplified stirrup footplate 40 is presented. As will be seen, stirrup footplate 40 has an elongated body, which is symmetrical about a central axis 42. At a heel end 44 (shown on the left-hand side of FIG. 2), a pair of lateral arms 46, 48 extend perpendicularly away from central axis 42. Heel end 44 also includes a heel extension 50 which extends in a rearward direction along central axis 42. Also included as part of stirrup footplate 40 is a front-end portion 52 which also extends along central axis 42 in a direction opposite heel end 44. In this embodiment, front end portion 52 is generally rectangular, with a portion that tapers to meet with a central waist portion 54. FIG. 2 also shows how central waist portion 54 is positioned immediately adjacent lateral arms 46 & 48, that will allow for appropriate support of the user's foot. As will be further discussed below, the configuration of this stirrup component is uniquely designed so that it will cooperate with other components of an AFO to provide previously unseen levels of stability. While the embodiment illustrated below includes particular proportions that have been found to be beneficial, it is contemplated that other variations are possible.

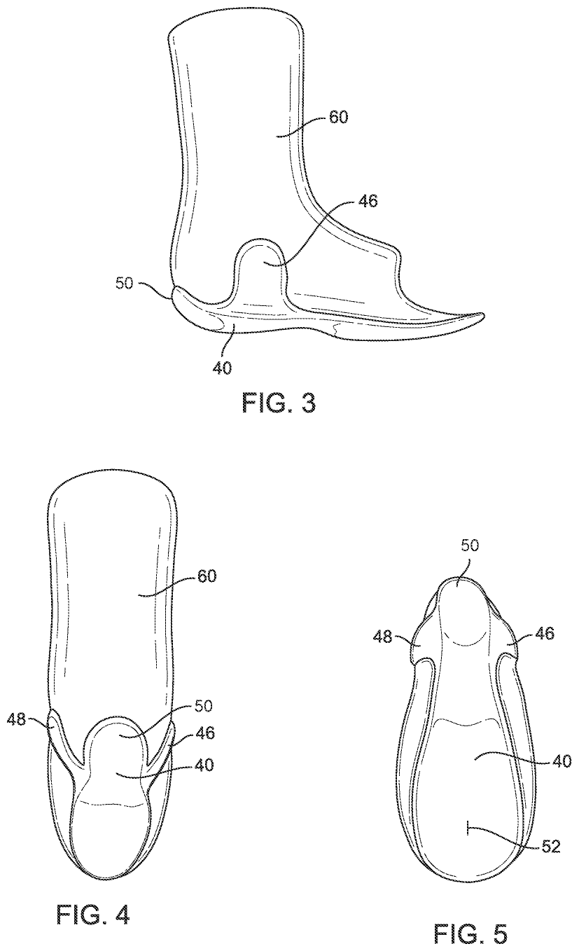

As suggested above, it is contemplated that stirrup footplate 40 may be heat bonded to a thermoplastic portion of an AFO 60. In one embodiment, this bonding will occur during a vacuum forming process which will produce a unitary component having additional levels of support for a user's foot. The FIGS. 3-5 below illustrate the results of this forming process. To provide support and stability for an AFO 60, stirrup footplate 40 is incorporated as bottom portion of AFO 60. As shown, heel end 44 of stirrup footplate 40 is positioned to be below the heel of a user. Stated differently, stirrup footplate 40 is molded to a bottom side of AFO 60 so that heel portion 44 is positioned so that a center point 45 of the rear portion 44 is generally aligned with the user's heel. Lateral arms 46 and 48 are then formed to surround and extend partially upward, thus wrapping or surrounding the bottom portion of a user's heel. Similarly, heel extension 50 will also extend upwardly, to surround the rear portion of a user's heel. As can be seen in FIGS. 3-5, this configuration is formed as part of AFO 60 where the heel portion of stirrup footplate 40 will form a cup or bowl like supporting structure which will surround and support for a user's heel when placed in AFO 60. Further, front end portion 52 will extend forward, and be situated below a footplate of AFO 60, thus also providing additional support for this portion of a user's foot.

It is generally contemplated that stirrup footplate 40 will be a formable plastic material, that can be easily formed into portion of AFO 60. That said, stirrup footplate 40 will be more rigid or stiff than material making up AFO 60, thereby providing specific levels of additional support for the portions of AFO 60 where this component is incorporated. Further, it is contemplated that various types of alternative materials could be used, but certain types of plastics are well suited for this purpose. Additionally, the thermoplastic material contemplated can also be combined with other components, such as padding and supporting straps, and other components used to form a completed AFO 60

As will be recognized, the stirrup footplate 40 reinforces (stiffens) the toe plate of AFO 60 and the lateral extensions 46, 48 on either side provide transverse (torsion) plane control. In its most general form, this stirrup footplate 40 is an addition to a lower extremity foot plate that provides additional features not previously possible. In most cases, it will be added to existing models of an AFO that are specifically tailored for a particular patient.

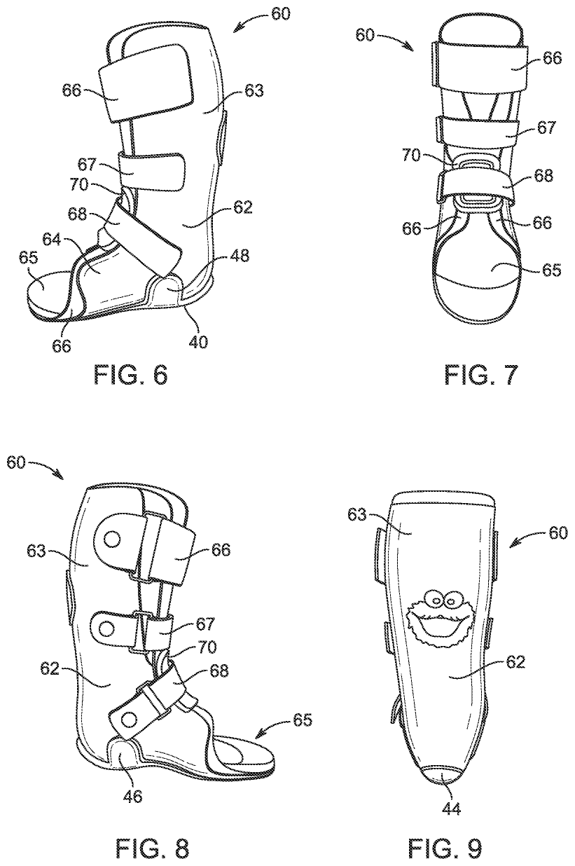

As suggested above, one application of the stirrup footplate 40 will allow an AFO 60 to be inserted into a shoe of the user. Generally, this is achievable by configuring the stirrup footplate 40 from a relatively thin portion of plastic material, which is easily formable to the bottom of an AFO's foot bed. One embodiment of AFO 60 is illustrated in FIGS. 6-11. FIGS. 6-9 show a first side, front, second side, and rear view, respectively, of completed AFO 60.

Referring now to FIGS. 6-9, AFO 60 is generally illustrated as having a main boot portion 62 which is designed and configured to surround a portion of a user's foot, ankle, and a portion of the lower leg. Specifically, AFO 60 has a foot portion 64, which will surround a portion of the user's foot, while also having an open toe portion 65, configured to allow the front foot and the toes of the user to extend beyond the foot portion. AFO 60 and specifically main body 62 has an upper portion 63 which is configured to surround the rear portion of a user's lower leg, specifically a portion of their calf. To form a proper enclosure for the user's foot, a number of straps are also included. In this embodiment, a first strap 66, a second strap 67, and a third strap 68 are all appropriately positioned to allow a user's leg and ankle to be captured within AFO 60. A front ankle pad 70 is also included, to provide support and comfort for the user. As can be seen in FIGS. 6-9, stirrup foot plate 40 is formed on the bottom portion of AFO 60 and an integral part thereof. As discussed above, stirrup foot plate 40 reinforces or stiffens the foot plate of AFO 60, well also providing torsional support.

It is important to note that the stirrup footplate 40 described and illustrated above is formed from a type of plastic, so that it can be easily bonded to a thermoplastic AFO 60. Although it is an additional component, it will provide the ability to be easily combined with the thermoplastic AFO 60 in a manner that essentially creates a unitary component. By carefully selecting the material thickness (i.e., the thickness of the plastic material making up the AFO 60), the level of added support can be customized to meet the specific needs of a patient. Additionally, the type of material used can also be varied to meet desired treatment goals. It is contemplated that the stirrup can be formed from polypropylene, or carbon infused polycarbonate, although other alternatives may be possible. Further, stirrup footplate 40 can be manufactured in many different ways, including but not limited to injection molding, 3D printing, stamping, etc. Further, it is possible that stirrup footplate 40 can be integrally formed as a portion of AFO 60 itself, and may undergo trimming after it has been bonded to AFO 60 to allow for customized fittings.

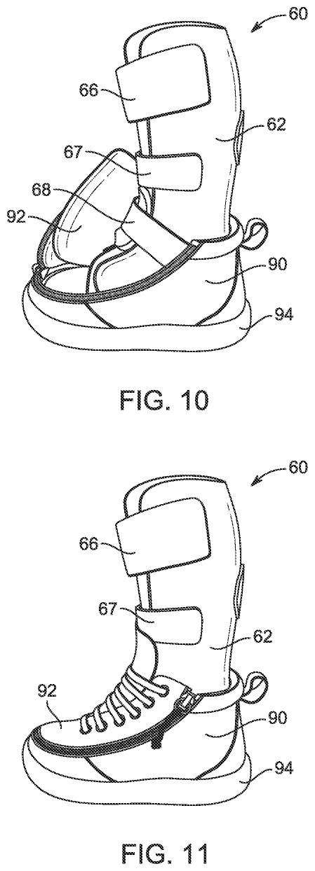

To provide ambulatory therapy and necessary support for patients, AFO 60 is specifically designed to be insertable into a shoe. Referring now FIGS. 10 and 11, an embodiment is illustrated wherein AFO 60 is insertable into a shoe 90, thus allowing a user to make use of AFO 60 in a number of different environments. This particular embodiment, shoe 90 has a zippered front enclosure 92 which is specifically designed to surround a portion of AFO 60. In addition, sole 94 is specifically configured to provide traction and support as a user traverse or walks around various surfaces or locations.

The poly carbonate stirrup comes in various sizes and thicknesses to accommodate pediatric and adult patients and to provide various levels of support. Various embodiments can allow for the use of plastic materials that are 3/32″, ⅛″, 5/32″, 3/16″ thick. Clearly, this can vary depending upon the specific type of material used and the level of stiffness desired. Clearly, other thicknesses are possible. Also, it is expected that the stirrup footplate 40 may be offered in many different sizes and slightly different configurations so that different sizes of patients can be accommodated. In addition, the material to be used will effect stiffness and the level of support provided. In one example, various poly-carbon materials have been found to be effective. Other material may include polypropylene, or carbon infused polycarbonate.

Various embodiments of the invention have been described above for purposes of illustrating the details thereof and to enable one of ordinary skill in the art to make and use the invention. The details and features of the disclosed embodiment[s] are not intended to be limiting, as many variations and modifications will be readily apparent to those of skill in the art. Accordingly, the scope of the present disclosure is intended to be interpreted broadly and to include all variations and modifications coming within the scope and spirit of the appended claims and their legal equivalents.

1. A stirrup footplate bondable to a thermoplastic Ankle Foot Orthosis (AFO) which is configured to be received in a shoe which is sized and configured to fit a user, the stirrup footplate comprising:
a plastic main body having a predetermined thickness and a generally elongated configuration which extends along a main axis, the main body further having a front end portion generally extending below a forefoot of the user when bonded to the AFO, the main body further having a heel end with a cross-like configuration.
wherein the cross-like configuration has a pair of lateral extensions extending substantially perpendicularly from the main axis and sized so that an end portion of each lateral extension will be formable in a manner to extend up an opposite side of the thermoplastic AFO when bonded thereto, the cross-like configuration further having a heel extension at a heel end of the main body which extends along the main axis, the heel extension configured to extend at least partially up a heel side of the AFO when bonded thereto; and
wherein the plastic main body is formed of a material having a predetermined stiffness which can be the same level of thickness and stiffness but more often is stiffer than the thermoplastic AFO which, when bonded to the thermoplastic AFO, will provide a support profile that provides a combined level of support to a user's heel and foot bed.

2. The stirrup footplate of claim 1 wherein the main body is formed of polypropylene and carbon infused polycarbonate.

3. The stirrup footplate of claim 1 or 2 wherein the predetermined thickness is in the range of 1/16 inch to ¼ inch.

4. The stirrup footplate of claim 1 wherein the main body has a length that is variable.

5. An ankle foot orthosis (AFO), comprising:
an AFO main body formed of a thermoplastic material comprising:
a heel section configured to surround a user's heel,
a footplate extending forwardly from a bottom portion of the heel section in a manner to support a forefoot of a user;
an upper section extending upwardly from the heel section an configured to substantially surround the user's heel and extending upward below the ankle on the medially and laterally; and
a closure mechanism coupled to the heel section to provide closure of the AFO around the user's ankle;
wherein the material forming the main body had a predetermined level of flexibility; and
a stirrup footplate coupled to a bottom side of the AFO, the stirrup footplate having a generally plate-like elongated configuration which extends along a main axis, the stirrup footplate further having a front end portion generally extending below the footplate of the AFO and having a heel end with a cross-like shape configured to be positioned below the heel section of the AFO, wherein the cross-like configuration has a pair of lateral extensions extending substantially perpendicularly from the main axis and sized so that an end portion of each lateral extension will extend up a side of the heel portion, the cross-like configuration further having a heel extension at the heel end extending along the main axis, the heel extension configured to extend at least partially up a rear portion of the heel section of the AFO;
wherein the stirrup footplate a plastic material having a predetermined stiffness which is stiffer than the thermoplastic material of the AFO, and wherein the stirrup footplate will provide a support profile that provides a combined level of support to the user's heel and footplate when the AFO is worn by a user.

6. The ankle foot orthosis (AFO) of claim 5 wherein the stirrup footplate has a predetermined thickness in the range of 1/16 inch to ¼ inch.

7. The ankle foot orthosis (AFO) of claim 6 wherein the stirrup footplate is formed of polypropylene and carbon infused polycarbonate.

8. The ankle foot orthosis of claim 5 wherein the pair of lateral extensions includes an outside extension and an inside extension, and wherein the outside extension extends upwardly to a location below a user's fibula making up the lateral malleolus and tibia making up the medial malleolus.

9. The ankle foot orthosis of claim 8 wherein the stirrup footplate is symmetric relative to the main axis and each of the lateral extensions are of the same length.

10. The ankle foot orthosis of claim 5 wherein the AFO main body is sized to fit within a shoe of the user.
